(19) United States
(12) Patent Application Publication
(10) Pub. No.: US 2011/0077558 A1
(43) Pub. Date: Mar. 31, 2011

(54) ULTRASOUND ENDOSCOPIC SYSTEM

(75) Inventors: Isaac Ostrovsky, Wellesley, MA (US); Russ Durgin, Bellingham, MA (US); Theresa Durgin, Bellingham, MA (US)

(73) Assignee: Boston Scientific Scimed, Inc.

(21) Appl. No.: 12/883,231

(22) Filed: Sep. 16, 2010

Related U.S. Application Data
(60) Provisional application No. 61/272,489, filed on Sep. 29, 2009.

(51) Int. Cl. A61N 7/00

(52) U.S. Cl. 601/2

(57) ABSTRACT

Embodiments of the invention include a device having an elongate section extending from a distal end to a proximal end, the elongate section may be configured to be inserted into a cavity of a body. The device may also include an end effector at the distal end of the elongate section. The end effector may have an end face. The end face may include a transducer configured to vibrate in response to an electric current. The elongate section may include a conduit configured to circulate coolant to the end effector.

### FIELD OF THE INVENTION

Embodiments of the present invention relate to an endoscopic system. In particular, exemplary embodiments of the present invention relate to endoscopes to perform image guided hemostasis. Embodiments of the present invention also cover methods of using such devices.

### BACKGROUND OF THE INVENTION

An endoscope is a flexible instrument introduced into the body for diagnostic or therapeutic purposes. Typically, these devices are inserted into the body through a natural opening, and are delivered to a work site inside the body through a body channel, such as, for example, the esophagus. Endoscopes are widely used for diagnostic and therapeutic purposes inside a body. There are many different uses for endoscopes, and typically, endoscope designs may be varied to optimize their performance for an intended application. For example, there are upper endoscopes for examination of the esophagus, stomach and duodenum, urethroscopes for examining the urethra and bladder, colonoscopes for examining the colon, angioscopes for examining the blood vessels and heart, bronchoscopes for examining the bronchi, laparoscopes for examining the peritoneal cavity, arthroscopes for examining joint spaces, sigmoidoscopes for examining the rectum and sigmoid colon, etc. Each of these devices may include features to optimize their performance for their application. Although embodiments of the current invention may be broadly applied to an endoscopic instrument used for any diagonostic or therapeutic procedure, for the sake of brevity, embodiments of the invention will be described as being applied to an endoscope especially configured to perform image guided hemostasis.

In typical applications, a distal end of an endoscope may be inserted into the body through a natural anatomic opening, such as, for example, the mouth, rectum, vagina, etc. In some embodiments, an endoscope may be inserted through an organ wall to access sites outside of a body lumen. In some embodiments, an endoscope may also be delivered percutaneously, through a trocar or a PEG tube, for example. The endoscope may be pushed into the body such that the distal end of the endoscope then proceeds from the point of insertion to a region of interest (work site) within the body by traversing a body channel. In the case of the exemplary embodiment described in this application, the work site may include a bleeding ulcer in the stomach. The endoscope may include one or more lumens extending longitudinally from the proximal end to the distal end of the endoscope. These lumens may deliver various diagnostic/treatment devices to the work site within the body. In some cases, an endoscopic device may be reused for different medical procedures after sterilizing the endoscope thoroughly after each procedure. Although sterilization of the endoscope may decrease the possibility of infection being transmitted from one patient to another, the potential for transmitting disease due to insufficient sterilization may still exist. Therefore, it may be desirable to develop a low cost single-use endoscope that is configured to perform a desired medical procedure.

### SUMMARY OF THE INVENTION

An embodiment of the invention may include an endoscopic device. The endoscopic device may include an elongate section extending from a distal end to a proximal end. The elongate section may be configured to be inserted into a cavity of a body, and may include an end effector at the distal end of the elongate section. The end effector may have an end face. The end face may include a transducer configured to vibrate in response to an electric current. The elongate section may also include a conduit configured to circulate coolant to the end effector.

Various embodiments of the invention may include one or more of the following aspects: the transducer may be configured to vibrate in response to an electric current at RF frequency; the transducer may be one of a dome shaped, pyramid shaped and a frustoconical shape; the transducer may be configured to vibrate in response to laser energy; a front side of the transducer may be configured to contact a body tissue and a back side of the transducer may be in fluid communication with the conduit; the back side may form a wall of a reservoir configured to receive the coolant; the end effector may include an inlet channel and an outlet channel in fluid communication with the reservoir; the end effector may include a thermoelectric cooler; the end effector may include an imaging device configured to provide an image of a region within the cavity; the end effector may have a substantially cylindrical shape having a first diameter at a first end and a second diameter at a second end, the first diameter may be smaller than the second diameter; a diameter of the end effector may transition step-wise from the first diameter to the second diameter at a location between the first end and the second end; the distal end of the elongate member may be disposed circumferentially outwards the first end of the end effector; the transducer may be made of a piezoelectric material; the transducer may include a coating of a metal; the transducer may include a coating of a Teflon based material; the transducer may include a non-stick coating material; the end effector may include a reservoir configured to maintain a supply of a coolant, an inlet channel conduit configured to deliver the coolant to the reservoir, and an outlet channel configured to remove the coolant from the reservoir; the elongate member may include a fluid conduit that mates with the inlet channel and a separate fluid conduit that mates with the outlet channel; the inlet channel and the outlet channel may be configured to fluidly couple to a heat exchanger; the inlet channel and the outlet channel may be configured to be fluidly coupled to a pump; the device may further including one or more control mechanisms that are configured to control an operation of the endoscope; the one or more control mechanisms may include a power supply configured to deliver an electric current at RF frequency to the transducer; the end face may include one or more illumination devices configured to illuminate the cavity; and the device may be used for visualization in conjunction with another visualization means.

An embodiment of the invention may include a method of using an endoscopic device. The method may include inserting a distal end of the device into a cavity of a body. The device may include an end effector having a transducer. The transducer may be configured to mechanically vibrate in response to an electric current. The method may also include pressing the transducer against tissue within the body, and vibrating the transducer against the tissue to heat the tissue. The method may further include circulating a coolant through the end effector to remove heat, and retracting the endoscope from the body.

Various embodiments of the invention may include one or more of the following aspects: pressing the transducer against tissue includes pressing a front side of the transducer against bleeding tissue; retracting the endoscope includes retracting the endoscope after stopping the bleeding; circulating the coolant includes flowing the coolant against a back side of the transducer; circulating the coolant includes circulating the coolant through the end effector within the body, and a heat transfer device outside the body; the method may further include obtaining a visual image of a region of the cavity using the imaging device; the transducer may be a piezoelectric based material; circulating the coolant may include circulating the coolant to prevent excessive heating of the tissue; the transducer may vibrate in response to an electric current at RF frequency; the device may be inserted into the body intraluminaly, transluminaly, or percutaneously; the heating of the tissue may be part of a tissue ablation process, a process to activate/melt devices, a process to fasten tissue, a process of vessel occlusion, or a process of disease treatment; and the method may also include using the imaging device in addition to a different imaging device for visualization.

An embodiment of the invention may also include a method of using a medical instrument. The method may include inserting the medical instrument into a body cavity. The medical instrument may include a transducer coupled to a distal tip thereof. The method may also include pressing the distal tip against an anatomic stricture within the body cavity, and activating the transducer to vibrate the distal tip and allow the medical instrument to traverse the stricture.

Various embodiments of the invention may also include circulating a coolant in the medical instrument to remove heat from the transducer; modulating a flow of coolant in the medical instrument to control a stiffness of the medical instrument; and activating the transducer includes activating the transducer using an electric current at RF frequency.

### BRIEF DESCRIPTION OF THE DRAWINGS

The accompanying drawings, which are incorporated in and constitute a part of this specification, illustrate embodiments of the invention and together with the description, serve to explain the principles of the invention.

FIG. 1 is a schematic view of an embodiment of an endoscope performing an exemplary endoscopic procedure.

FIG. 2 is an illustration of the endoscope of FIG. 1.

FIGS. 3A and 3B are isometric and cross-sectional illustrations of an end effector of the endoscope of FIG. 2.

FIG. 4 is an illustration of a method of using an embodiment of the current invention.

### DESCRIPTION OF THE EMBODIMENTS

Reference will now be made to exemplary embodiments of the invention, examples of which are illustrated in the accompanying drawings. Wherever possible, the same reference numbers will be used throughout the drawings to refer to the same or like parts.

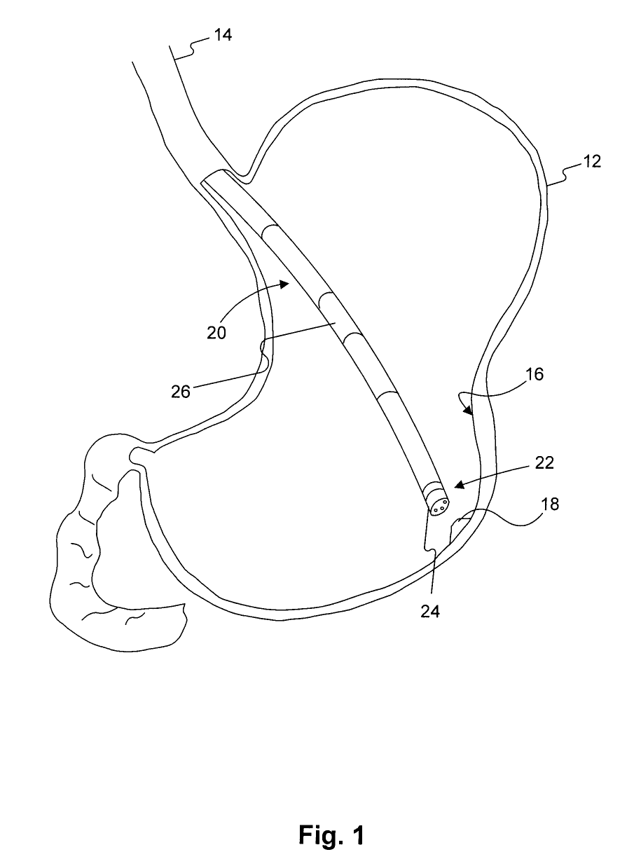

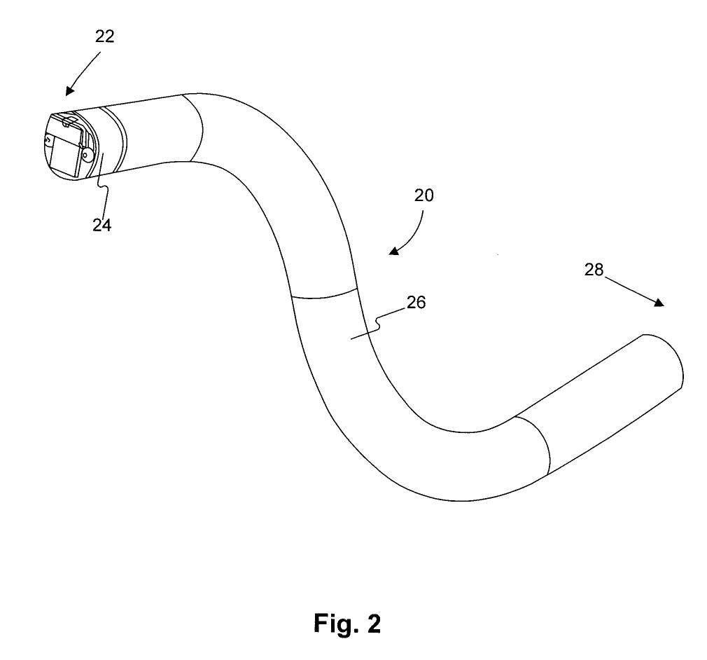

FIG. 1 depicts an exemplary endoscope 20 performing an exemplary hemostasis procedure of a peptic ulcer 18 on a patient. Endoscope 20 may be inserted into stomach 12 through the esophagus 14, and positioned in stomach 12 such that a distal end 22 of endoscope 20 may be positioned proximate a peptic ulcer 18 (work site) on stomach wall 16. A proximal end 28 (see FIG. 2) of endoscope 20 may extend out of the body of the patient and may be controlled to perform hemostasis of ulcer 18. It should be emphasized that the medical procedure illustrated in FIG. 1 is exemplary only, and that endoscopes of the current disclosure may be applied to any endoscopic application known in the art. It should also be emphasized that the illustration of the endoscope in FIG. 1 is exemplary only, and that endoscopes 20 of the current disclosure may also be guide tubes, catheters or the like without limitation.

FIG. 2 illustrates endoscope 20 used in the hemostasis procedure of FIG. 1. Endoscope 20 may include a flexible elongate member 26 extending between the proximal end 28 and distal end 22. Elongate member 26 may be flexible so as to enable endoscope 20 to bend and pass through tortuous body passages as distal end 22 of endoscope 20 advances to the location of ulcer 18 (see FIG. 1). The proximal end 28 of endoscope 20, in some embodiments, may include an actuation device configured to operate endoscope 20. Distal end 22 of endoscope 20 may include an end effector 24 that may include devices/instrumentation suited for hemostasis of ulcer 18. End effector 24 may be operatively coupled to, and controlled by, the actuation device. In some embodiments, in place of a discrete actuation device, cables and conduits extending from the proximal end 28 may be coupled to other devices, such as power supplies, pumps, and visual displays.

Elongate member 26 may include a plurality of lumens running longitudinally therethrough. These lumens may extend between distal end 22 and proximal end 28. These lumens may serve as conduits for supplying electrical signal, suction, fluid, light, vision, and/or other supplies to distal end 22 and end effector 24.

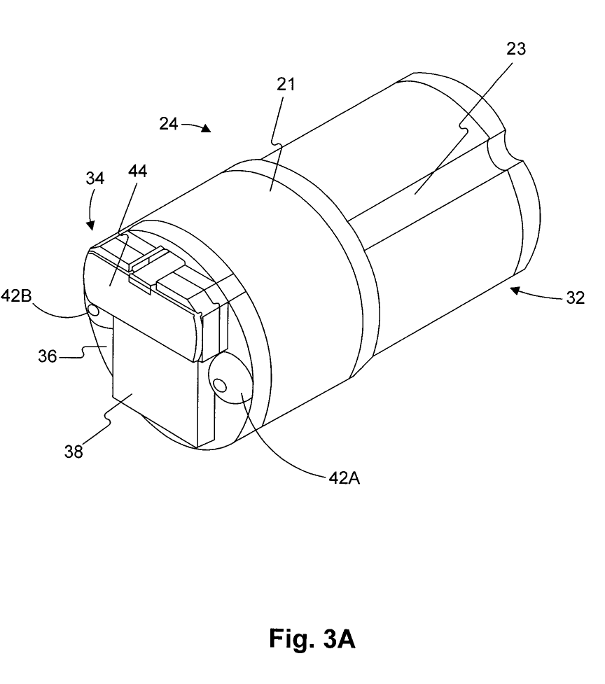

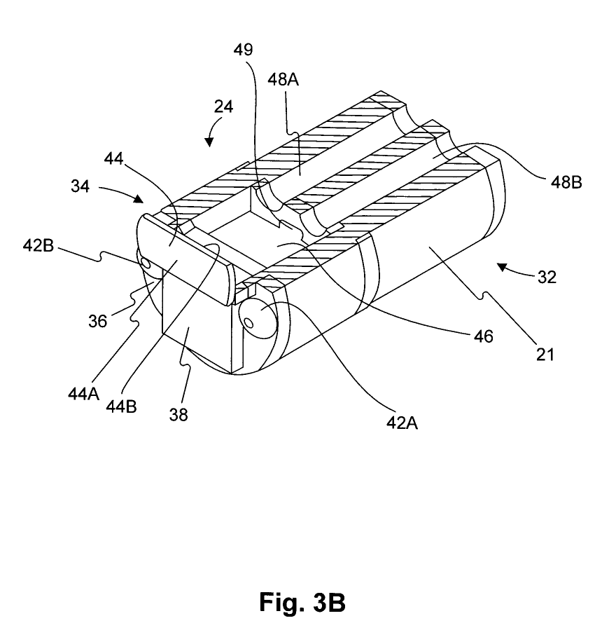

FIGS. 3A and 3B illustrate embodiments of end effector 24 that may be coupled to distal end 22 of endoscope 20. FIG. 3A illustrates an isometric view, and FIG. 3B illustrates a cross-sectioned view of the end effector 22. In the discussion that follows, reference will be made to both FIGS. 3A and 3B. Although end effector 24 is depicted as having a substantially cylindrical external shape with an end face 36 at one end, it is contemplated that end effector 24 may have any other shape, without limitation.

In some embodiments, end effector 24 may include a housing 21 that extends from a proximal end (hereinafter referred to as “first end 32”) having a first diameter to a distal end (hereinafter referred to as “second end 34”) having a second diameter, larger than the first diameter. The external diameter of the housing 21 may transition stepwise from the first diameter to the second diameter at a location between the first and the second end 32, 34. The first end 32 of housing 21 of end effector 24 may be coupled to a distal end of elongate member 26, and the second end 34 may be coupled to the end face 36. In some embodiments, the distal end of elongate member 26 may be slid over the first end 32 of housing 21 such that an internal surface of the distal end of elongate member 26 may mate with, and slide over, an external surface of the first end 32 of housing 21. In some embodiments, key ways or slots 23 on external surface of first end 32 may mate with corresponding features on the internal surface of the distal end of elongate member 26, to align conduits in housing 21 to respective mating conduits in elongate member 26. In the assembled configuration (shown in FIG. 2), endoscope 20 may be substantially tubular having a substantially uniform external diameter from proximal end 28 to distal end 22.

End face 36 may be fixedly or removably attached to housing 21, and may include an imaging device 38 coupled thereto. Imaging device 38 may be configured to deliver a video image of an area in front of distal end 22 of endoscope 20. This video image may enable endoscope 20 to be inserted and positioned proximate ulcer 18, and perform hemostasis of ulcer 18 guided by the image. Although any device (such as, a CCD camera) capable of obtaining an image of the work site within the body may be used as imaging device 38, in some embodiments, imaging device 38 may include a CMOS video chip and/or an optical fiber. One or more lumens extending through elongate member 26 may include cables that deliver electrical supply to power the imaging device 38, and cables that deliver signals from imaging device 38 to proximal end 28 of endoscope 20. These cables may be electrically coupled to video monitors or other display devices that are configured to display an image of the work site thereon.

Illumination devices 42A and 42B (such as bulbs, solid state light emitting devices (LEDs), etc.) may also be coupled to end face 36 to illuminate the work site. Cables, coupled to these illumination devices 42A and 42B, may also extend to proximal end 28 through lumens passing through elongate member 26, to deliver electrical power and other signals to these illumination devices 42A and 42B. Although FIGS. 3A and 3B depicts two illumination devices 42A and 42B positioned on either side of imaging device 38, it is contemplated that other embodiments of endoscope 20 may include a different number of illumination devices arranged in different patterns on end face 36.

End face 36 may also include a transducer 44 coupled thereto. Transducer 44 may have any shape, geometry, and dimension (for example, dome shaped, pyramid shaped, frustoconical shaped, etc.) to suit the treatment. Transducer 44 may include a high intensity ultrasound transducer. Transducer 44 may be made of a piezoelectric ceramic material (such as, for example, PZT-8), which may respond mechanically to an electrical excitation. Transducer 44 may include a front-side 44A and a back-side 44B spaced apart from each other. In some embodiments, front-side 44A and a back-side 44B may be disposed substantially parallel to each other. One or both of front-side 44A and back-side 44B of transducer 44 may be plated with a metal (such as, for example, silver, gold, nickel, etc.). In some embodiments, front-side 44A may alternatively or additionally be coated with a biocompatible material that may reduce stickiness of transducer 44 to body tissue (such as, for example, a Teflon based or other non-stick coating). In response to an electric current of RF frequency (such as, for example, from about 3 MHz-20 MHz), transducer 44 may generate mechanical vibrations. When front-side 44A of transducer 44 is pressed against body tissue, these mechanical vibrations may penetrate into tissues. The tissues may absorb these mechanical vibrations and generate heat. When transducer 44 is pressed against a bleeding ulcer 18, the heat and the pressure may make the walls of the surrounding tissue locally fuse together to seal ulcer 18 and surrounding blood vessels to stop the bleeding. Although transducer 44 is described in a procedure to seal ulcer 18, in general, transducer 44 may be applied in any procedure to apply heat to an area. For example, transducer 44 may be applied in a procedure to provide heat to ablate tissue, activate/melt devices (fasteners, etc.), fasten tissue, vessel occlusion (cancer, etc.), disease treatment, etc.

To prevent over-heating of the surface of the body tissue at the contact area, and to inhibit heat related effects on transducer 44 and surrounding devices, transducer 44 may be cooled from the back-side 44B. Cooling of transducer 44 may be achieved by keeping back-side 44B of transducer 44 in contact with a circulating coolant. Back-side 44B may form a wall of a reservoir 46, enclosed by housing 21, and configured to maintain a supply of the coolant in contact with back-side 44B. The coolant may be circulated through reservoir 46 to transfer heat from distal end 22 within the body to proximal end 28 outside the body. At proximal end 28, the coolant may be circulated through other devices, such as, a heat exchanger and a pump, which may help in circulating a cool coolant through reservoir 46. An inlet channel 48A may deliver the coolant from proximal end 28 to reservoir 46, and an outlet channel 48B may deliver the coolant from reservoir 46 to proximal end 28. Inlet channel 48A and outlet channel 48B may comprise the lumens running longitudinally through elongate member 26.

Any fluid (such as, for example, water, saline, oils, refrigerants, etc.) may be used as coolant. Although a liquid may typically be used as coolant, it is contemplated, that in some embodiments, a gaseous coolant may be used to cool transducer 44. Using the coolant to prevent over heating of transducer 44 may reduce the likelihood of tissue charring and may extend the working life of transducer 44. Although the description above, and FIG. 3B, depicts the back-side 44B of transducer 44 as forming a wall of reservoir 46, other configurations are also contemplated. For example, in some embodiments, a discrete reservoir 46 may be eliminated, and heat from transducer 44 may be transferred to the coolant passing from a continuous inlet channel 48A to outlet channel 48B. Cooling of transducer 44 may also be affected or enhanced with a thermoelectric module situated in the vicinity of transducer 44.

In addition to inlet channel 48A and outlet channel 48B, housing 21 of end effector 24 may also include other channels 49 that may deliver cables coupled to the illumination devices 42A and 42B, imaging device 38, transducer 44, and any other device on end face 36 to the actuation device or other control devices on proximal end 28. The actuation device (or the other control devices) may include controls that control the operation of endoscope 20. These controls may include switches or knobs that activate the devices (such as, illumination devices 42A and 42B, imaging device 38, transducer 44) coupled to end face 36, and initiate and regulate the amount of coolant circulated through end effector 24.

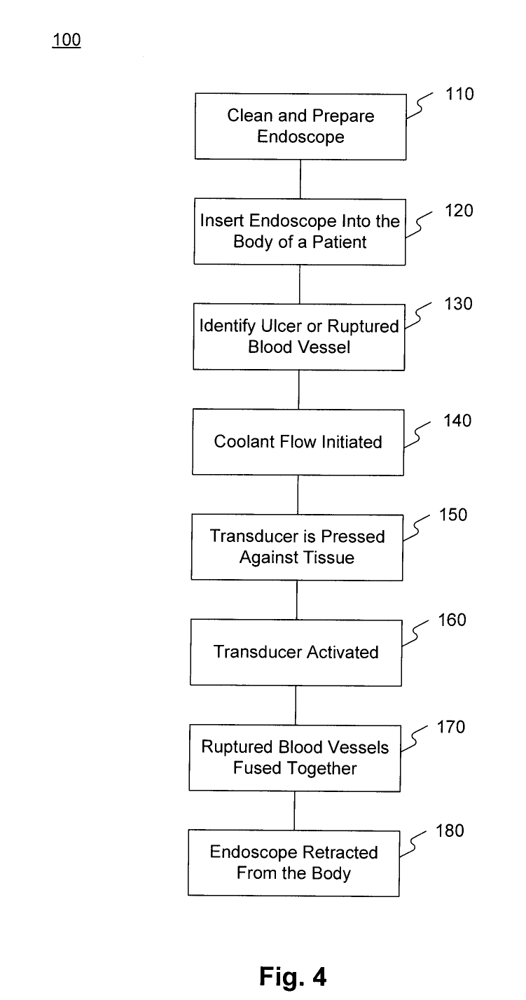

A method 100 of using endoscope 20 in an exemplary hemostasis procedure of tissue, for example ulcer 18 of FIG. 1, will now be described. FIG. 4 is a flow chart that illustrates the steps involved in the exemplary hemostasis procedure. Endoscope 20 may be prepared to be inserted into a body cavity (step 110). In some embodiments, this step may include cleaning endoscope 20 and/or coupling end effector 24 to the distal end of elongate member 26. Typically end effector 24 may be permanently affixed to elongate member 26 during manufacture. The external surfaces of endoscope 20 may also be lubricated for ease of insertion into a body cavity. In some embodiments, this step may also include fluidly coupling inlet and outlet channels 48A, 48B to pumps and heat exchangers, and electrically coupling cables from illumination devices 42A, 42B, imaging device 38, and transducer 44 to control devices. These control devices may include visual monitors electrically coupled to imaging device 38, and power supplies electrically coupled to illumination devices 42A, 42B, and transducer 44. The power supply coupled to transducer 44 may be configured to direct an electric current at RF frequency to transducer 44.

The distal end 22 of endoscope 20 may be inserted into the mouth of the patient and pushed down the body of the patient through the esophagus 14 (step 120). The illumination devices 42A, 42B and imaging device 38 may be activated, and the endoscope 20 inserted into the body guided by the image of the body cavity obtained by the imaging device 38. The image may help identify an ulcer 18 or a ruptured blood vessel within the body (step 130). In some embodiments, coolant flow through the endoscope 20 may now be initiated (step 140) and regulated to control the flexibility of the endoscope 20 during insertion. To reduce the stiffness of elongate member 26 as the endoscope 20 traverses through tortuous body cavities, coolant may be evacuated from endoscope 20. In cases where an increased rigidity of endoscope 20 is desired (for example, to push through an anatomical stricture in a body cavity), a controlled amount of coolant may be circulated through the endoscope 20. Increasing the amount of coolant in endoscope 20 may increase the rigidity of endoscope 20. In some embodiments, a thermoelectric module may be used instead of or in conjunction with the coolant. Guided by the image from imaging device 38, end face 36 of end effector 24 may be positioned in stomach 12 such that a distal end 22 of endoscope 20 may be positioned proximate ulcer 18 on stomach wall 16.

The endoscope 20 may be further pushed into the body so that the front-side 44A of transducer 44 may be pressed against ulcer 18 (step 150). With the transducer 44 firmly pressed against ulcer 18, the transducer 44 may be activated to induce a mechanical vibration on transducer 44 (step 160). The mechanical vibration of transducer 44, as the transducer is pressed against ulcer 18, may penetrate and heat up the tissue surrounding ulcer 18. The heat and the pressure of transducer 44 against the tissue may locally fuse the blood vessels surrounding ulcer 18 together, thereby sealing ruptured blood vessels, and stopping the bleeding (step 170).

As the tissue heats up, the coolant flow through the end effector may remove excess heat produced by the vibration of transducer 44 against body tissue, to prevent charring of the body tissue. The coolant in reservoir 46 may get heated due to the heat conducted from the back-side 44B of transducer 44. The hot coolant may be directed towards the proximal end 28 of endoscope 20 through outlet channel 48B. At the proximal end 28, outlet channel 48B may be fluidly coupled to a heat exchanger to dissipate the heat of the coolant. The cooled coolant may then be pumped back to reservoir 46 through inlet channel 48A, using a pump fluidly coupled to the circuit. The flow of coolant through reservoir 46 may thus cool transducer 44 and prevent excessive heating of transducer 44 and the tissue. The amount of coolant circulated through the endoscope 20 may be controlled to maintain a desired temperature of transducer 44.

After the ruptured blood vessels of ulcer 18 have been fused together, the front-side 44A of transducer 44 may be backed off of ulcer 18, and the endoscope 20 slowly retracted from the body of the patient (step 180). Although FIG. 4 illustrate the steps being performed one after the other in a serial manner, in some embodiments, the order of steps may be different, and some or all of these steps may be performed in parallel. For instance, in some embodiments, the transducer 44 may be activated (step 160) before front-side 44A of the transducer 44 is pressed against the ulcer (step 150). In some other embodiments, coolant flow may be initiated (step 140) along with activation of the transducer (step 160) and pressing of the transducer 44 against the ulcer 18 (step 150).

As indicated earlier, although an embodiment of the invention of the current disclosure is illustrated and described as being applied to an endoscope configured for hemostasis of a peptic ulcer, the invention may be broadly applied to any endoscopic or other suitable medical device. For instance, in some embodiments, a transducer 44 that vibrates in response to an electric current or other energy (such as, for example, laser energy) may be constructed as an add-on device. This add-on device may be coupled to the distal end of any catheter or like medical device to apply heat to an area to aid in any medical procedure. For example, transducer 44 may be used to deliver controlled focused heating to an area to aid in tissue ablation, activate/melt tissue devices such as fastening devices, disease treatment, etc. For instance, in the case of a biliary stricture, transducer 44 may be coupled (or embedded) in a distal tip of a duodenoscope, in the case of a colonic or esophageal stricture, transducer 44 may be coupled to the distal tip of a colonoscope or a gastroscope, respectively. Activation of transducer 44 may vibrate the distal tip of the catheter, endoscope, or other device as it tries to traverse a lesion in the body cavity. Such a device may also include a coolant being circulated past the transducer to remove the heat produced by the transducer. Vibration of the distal tip of the device may enable the device to more easily traverse the constricted body cavity proximate the lesion with minimal trauma to body tissue, thereby improving the procedural outcome and reducing the time needed to perform the procedure. In some embodiments, one or more transducers may also be coupled in different orientations (transversely, laterally, different angles, etc.) at a distal end of the endoscope to enable the endoscope to traverse constricted body cavities.

In another embodiment, a transducer 44 coupled to a transluminal device, such as an endoscope or a catheter, may be used as a mixing or a curing tool for injected or sprayed components. As a mixing tool, a two-part adhesive, delivered to a work site within the body, may be mixed by vibrating the mixture with transducer 44. As a curing tool, a patch attached over a puncture using a heat curable adhesive may be cured by pressing a vibrating transducer over the patch. The combination of the pressure and the heat produced by the vibrating transducer may assist in the curing of the adhesive.

Conventional endoscopes are expensive reusable devices. Most conventional endoscopes include an imaging system (fiber-optic imaging bundle, fiber-optic illumination, etc.), which may account for most of the cost of the endoscope, and one or more working channels. In these conventional endoscopes, the imaging system and working channels may also take most of the room within the body of the endoscope and the front plane of the distal tip. In an embodiment of the endoscope of the current disclosure, the endoscope may be a disposable device that may include an inexpensive CMOS chip instead of the more expensive imaging fiber-optic bundle, and may include an ultrasound transducer as part of the endoscope. In some embodiments, such an endoscope may also not have any working channels and may be dedicated for the hemostasis procedure. In some embodiments, an endoscope of the current disclosure may be used for imaging in addition to the traditional means. In some embodiments of the current disclosure, an ultrasound transducer may be provided on a catheter which may be delivered through a working channel of a conventional endoscope.

The embodiments described herein are exemplary only, and it will be apparent to those skilled in the art that various modifications and variations can be made in the disclosed systems and processes without departing from the scope of the invention. Other embodiments of the invention will be apparent to those skilled in the art from consideration of the specification and practice of the invention disclosed herein. It is intended that the specification and examples be considered as exemplary only, with a true scope of the invention being indicated by the following claims.

What is claimed is:

1. A device comprising:
an elongate section extending from a distal end to a proximal end and configured to be inserted into a cavity of a body, the elongate section including:
an end effector at the distal end of the elongate section, the end effector including an end face, the end face including a transducer configured to vibrate in response to an electric current; and
a conduit configured to circulate coolant to the end effector.

2. The device for claim 1, wherein the transducer is configured to vibrate in response to an electric current at RF frequency.

3. The device of claim 1, wherein the transducer is configured to vibrate in response to laser energy.

4. The device of claim 1, wherein a front side of the transducer is configured to contact a body tissue and a back side of the transducer is configured to be cooled by the coolant.

5. The device of claim 4, wherein the end effector includes a reservoir and the back side may form a wall of a reservoir configured to receive the coolant.

6. The device of claim 5, wherein the end effector includes an inlet channel and an outlet channel in fluid communication with the reservoir.

7. The device of claim 6, wherein the elongate member includes a conduit that mates with the inlet channel and a separate fluid conduit that mates with the outlet channel.

8. The device of claim 1, wherein the end effector includes an imaging device configured to provide an image of a region within the cavity.

9. The device of claim 1, wherein the end effector includes a substantially cylindrical shape having a first diameter at a first end and a second diameter at a second end, the first diameter being smaller than the second diameter.

10. The device of claim 1, wherein the transducer includes a coating of at least one of a metal or a non-stick material.

11. A method of using an endoscopic device, comprising:
inserting a distal end of the device into a cavity of a body, the device including an end effector having a transducer, the transducer being configured to mechanically vibrate in response to an electric current;
pressing the transducer against tissue within the body;
vibrating the transducer against the tissue to heat the tissue; and
circulating a coolant through the end effector to remove heat.

12. The method of claim 11, wherein pressing the transducer against tissue includes pressing a front side of the transducer against bleeding tissue.

13. The method of claim 11, wherein circulating the coolant includes flowing the coolant against a back side of the transducer.

14. The method of claim 11, wherein circulating the coolant includes circulating the coolant through the end effector within the body and a heat transfer device outside the body.

15. The method of claim 11, wherein vibrating the transducer includes directing electric current at RF frequency to the transducer.

16. The method of claim 11, wherein inserting a distal end of the device into a cavity of a body includes inserting the distal end into the cavity intraluminally, transluminally, or percutaneously.

17. A method of using a medical instrument, comprising:
inserting the medical instrument into a body cavity, the medical instrument including a transducer coupled to a distal tip thereof;
pressing the distal tip against an anatomic stricture within the body cavity; and
activating the transducer to vibrate the distal tip and allow the medical instrument to traverse the stricture.

18. The method of claim 17, further including circulating a coolant in the medical instrument.

19. The method of claim 18, further including modulating a flow of coolant in the medical instrument to control a stiffness of the medical instrument.

20. The method of claim 17, wherein activating the transducer includes activating the transducer using an electric current at RF frequency.
